(19) United States
(12) Patent Application Publication
(10) Pub. No.: US 2024/0082045 A1
(43) Pub. Date: Mar. 14, 2024

(54) URINATION FUNNEL FOR USE WITH MEDICAL SUCTION CANISTER SYSTEMS

(71) Applicants: Randal P. Ching, Bend, OR (US); Daniel R. Baker, Seattle, WA (US)

(72) Inventors: Randal P. Ching, Bend, OR (US); Daniel R. Baker, Seattle, WA (US)

(21) Appl. No.: 18/461,756

(22) Filed: Sep. 6, 2023

Related U.S. Application Data
(60) Provisional application No. 63/375,048, filed on Sep. 8, 2022.

(51) Int. Cl. A61F 5/44; A61F 5/451; A61M 1/00

(52) U.S. Cl. CPC A61F 5/4404; A61F 5/451; A61M 1/71

(57) ABSTRACT

A urination funnel for receiving and transporting urine voided by a user into a suction canister system includes an inlet for receiving the urine voided by the user. The inlet is configured to conform in shape to an anatomy of the user around a urethra of the user. The urination funnel also includes an outlet for transporting the urine voided by the user to the suction canister system. The outlet is configured to engage with a suction hose of the suction canister system. The urination funnel also includes a contoured funnel body extending between the inlet and the outlet.

### PRIORITY CLAIM

This application claims priority to and the benefit of U.S. Provisional Application with Ser. No. 63/375,048 filed on Sep. 8, 2022, entitled “PeeFunnel for Female Urination Assistance”, which is herein incorporated by reference in its entirety.

### FIELD OF INVENTION

The present application relates generally to medical devices and suction canister systems for removing human secretions using suction and, more specifically, for removing urine from a patient using suction applied to an external apparatus.

### BACKGROUND OF THE INVENTION

According to the Agency for Healthcare Research and Quality, each year, somewhere between 700,000 and 1,000,000 people in the United States fall in the hospital. Many injuries to hospitalized patients (as well as those in skilled nursing facilities and under home care) occur when falling on their way to use the toilet. “[Of the falls while ambulating,] the most frequent destinations were: bed to bathroom (37%), bedside commode to bed (11%), and bed to bedside commode (6%).” [Hitcho, 2004. J Gen Intern Med 19(7): 732-739]. Historically, there has been a large and generally unmet need for providing women with assistance when they are unable or if is unsafe for them to ambulate or use the toilet.

Bed pans have been around for many years in similar forms (e.g., U.S. Pat. No. 651,310 to Hogan titled, “Bed or douche pan”), which essentially are variously shaped bowls placed below the patient needing to micturate. Urine splashes can be challenging to control with these types of bed pans, and they are not designed for use by females. This led to bedside commodes (e.g., U.S. Pat. No. 210,917 to Clum titled “Improvement in portable closets or commodes”). However, while these devices allowed for urination closer to the bed (or patient location), they did not remove or reduce the need to transfer or position the whole body of the patient carefully. Additionally, these devices need to be emptied by an attendant (e.g., U.S. Pat. No. 815,481 to Smith titled, “Bed attachment”). That is, bedside commodes require similar movements as using a toilet, and since they do not flush, require regular maintenance to keep sanitary.

Moreover, bedpans, urinals, and commodes are cumbersome and unwieldly (especially for women).

Early attempts at providing more direct contact with the patient via external application and passively using gravity to move urine from that patient and into a container made incremental improvements (e.g., U.S. Pat. No. 3,374,790 to Mayhorne titled, “External female urinal”). Indwelling catheters (short- or long-term), placed internally into the bladder via the ureter, require strict adherence to cleaning and sanitation requirements to reduce infection, much maintenance time, and are uncomfortable for many patients (e.g., U.S. Pat. No. 2,455,859 to Foley titled, “Artificial sphincter and method”). Other systems function based on gravity or portable, battery-powered, self-contained pumps. In general, although bedpans, commodes, female urinals, and urinary catheters offer some benefits for female urination, none of these prior solutions are ideal, and catheterization poses a significant risk of infection and possibly injury (e.g., narrowing of the urethra due to scarring and bladder injuries resulting from improper insertion).

More recently, several urine collection systems have been invented that offer continuous, non-invasive, urine collection via negative pressure “wicking”, such as those described in U.S. Pat. No. 10,952,889 to Newton, et al., and U.S. Pat. No. 10,857,025 to Davis, et al. The wicking is provided by low pressure suction applied to these devices which are placed on, or near the ureter, and left in place. These devices are designed for longer-term use (on the order of many hours, or overnight) and can be helpful for those patients who are sedated or comatose. While these external catheter systems carry less risk of infection over internal catheters, their primary disadvantage over other traditional female urinary assistance devices or systems is they can be uncomfortable, less hygienic, and long-term urine contact with skin may cause dermal breakdown. Moreover, these external catheter systems normally contain several different types of plastic, either glued or heat-welded together, which cannot easily be separated. Not all the plastic types are recyclable and, those that are, cannot be easily separated from those that are not plus these designs take additional time to separate and sort.

A funnel-shaped urine collector, integrated with a urine collection system, is described in U.S. Pat. No. 6,311,339 to Kraus. The urine collector described therein is intended for use by both males and females with slight variations in shape to accommodate anatomical differences. However, both the male and female funnel designs incorporate the use of a “urine collection well” located at the bottom of a polymeric funnel-shaped collector (bottom being the lowest point with respect to gravity). This practically limits the urine collector to a person in the supine position because the system can only function when gravity pools the urine into the “urine collection well” (i.e., for a person in a seated position, the urine collection well would no longer be at the lowest point with respect to gravity). Furthermore, the tubing/hose that connects the funnel-shaped collector to the vacuum tank enters the funnel at the uppermost portion of the funnel-shaped collector (highest point with respect to gravity) requiring that the tubing/hose run inside the collector with the tip positioned at the bottom of the “urine collection well.” This design could add to patient discomfort with the device. Additionally, there is no provision to secure the tubing running inside of the funnel-shaped collector so that it could become accidentally displaced (dislodged) from the collection well while in use. When removing the hose, the hose will have urine on its outside making cleaning more complex and time-consuming. Also disclosed is the use of a manually-operated “vacuum control orifice” at the top of the funnel to adjust the air flow and vacuum pressure in the collector.

Hence, an inexpensive, disposable (biodegradable and/or compostable), non-invasive, and hygienic solution for assisting women to urinate while receiving care or without having to get out of bed to use the toilet is needed. The patent literature includes various systems for collecting or transporting urine, such as U.S. Pat. No. 4,904,248 to Vaillancourt, U.S. Pat. No. 6,736,803 to Cawood, U.S. Pat. No. 7,018,366 to Easter, U.S. Pat. No. 7,135,012 to Harvie, and U.S. Patent Publication No. 2011/0054426 A1 to Stewart. In general, these systems function based on gravity (Vaillancourt, Cawood, and Stewart) or portable, battery-powered, self-contained pumps (Easter and Harvie) and do not involve the use of a funnel connected to a standard medical suction canister system. A number of funnel-shaped receptacles for collecting and conveying urine from females are also disclosed in U.S. Pat. No. 3,964,111 to Packer, U.S. Pat. No. 7,181,781 to Trabold, and U.S. Patent Publication No. 2014/0325746A1 to Block. These relate to funnel-shaped receptacles that assist females in urinating either gravity (Packer and Trabold) or with vacuum assistance via an integrated bellows (non-continuous suction) pump (Block). Once again, however, no accommodation is made for using these funnels with standard medical suction canister systems.

### SUMMARY

Described herein is a urination funnel having an inverted funnel design such that tubing connecting the urination funnel to a suction canister connects with a lowest part of the urination funnel (with respect to gravity) rather than the tubing entering the urination funnel at its highest point and traveling inside to the collection well, as provided in prior solutions. This design eliminates the need for any “urine collection well” and allows the tubing to be connected (press-fit) to the outside of the urination funnel instead of having to be run inside the urination funnel to the collection well. This provides several distinct advantages of the urination funnel compared to those of prior designs. Specifically, the overall funnel size and volume may be decreased since the tubing does not need to run inside the urination funnel to a collection well, allowing the urination funnel to more easily and comfortably fit between the legs of the user. Also, as the tubing may be “press-fit” directly onto the tapered cylindrical outlet nozzle, the tubing may be firmly secured to the urination funnel, preventing the tubing from becoming detached during use. Moreover, unlike in prior designs, only an internal surface of the tubing will be in contact with urine, making clean up easier and faster. As the tubing connects to the urination funnel at its lowest point (with respect to gravity), this enables the user to urinate in multiple anatomical positions (from standing, to seated, to lying down), rather than just supine which can serve far more users. Finally, unlike in prior designs, the urination funnel need not remain in in the upright position so that urine remaining in the well does not leak out.

Another feature of the urination funnel described herein is the use of molded pulp material for the urination funnel instead of the “polymeric” material typically used in prior solutions. For example, the urination funnel may be manufactured from a molded pulp material which can be combined in many ways, orientations, and amounts, and include wood fiber, recycled newsprint, bagasse (sugarcane), bamboo, wheat, straw, corn, fungal mycelium or fungal-based substrate, and other plant-based fibers. These molded pulp materials can be made to be porous, allowing the passage of air, while remaining water-resistant (similar in concept to “breathable” water-resistant fabrics). Moreover, the urination funnel may be manufactured for reusable applications from materials such as plastic, metal, or composites, in which a pre-sized vent hole could be incorporated on the upper surface of the urination funnel (upper with respect to gravity) to enable automatic air flow and vacuum pressure adjustment according to the vacuum pressure set by the suction canister system and potentially by patient mediated covering of the hole. These designs eliminate the need for the manually operated “vacuum control orifice”, affords an airflow pattern which is more likely to remove leakable urine out of the urination funnel after use, and, due to its biodegradable/compostable nature, favors sustainable manufacturing while reducing plastic environmental waste.

The urination funnel disclosed herein is designed for use with existing standard suction canister systems rather than being part of a custom integrated vacuum subsystem. The geometry of the urination funnel outlet has been sized to fit medical industry standard (off-the-shelf) suction canister tubing so the tubing can be easily connected (press-fit) onto the funnel. These low-vacuum-pressure suction canister systems are currently ubiquitous in hospitals, rehab centers, and nursing homes for nasogastric (NG), airway/mucus suctioning, and other uses thus eliminating the need for a custom vacuum subsystem.

According to an aspect of the present disclosure, a urination funnel for receiving and transporting urine voided by a user into a suction canister system is provided. The urination funnel includes an inlet for receiving the urine voided by the user, wherein the inlet is configured to conform in shape to an anatomy of the user around a urethra of the user. The urination funnel also includes an outlet for transporting the urine voided by the user to the suction canister system, wherein the outlet is configured to engage with a suction hose of the suction canister system. The urination funnel also includes a contoured funnel body extending between the inlet and the outlet.

According to an embodiment of any paragraph(s) of the present disclosure, the inlet includes a rolled lip that is configured to conform in shape to the anatomy of the user around the urethra of the user.

According to an embodiment of any paragraph(s) of the present disclosure, a material of the urination funnel is a molded pulp material from plant-based fiber material.

According to an embodiment of any paragraph(s) of the present disclosure, the plant-based fiber material is recycled.

According to an embodiment of any paragraph(s) of the present disclosure, the molded pulp material has optimized fiber length, fiber orientation, binder, and wall thickness to provide sufficient structural rigidity to support low vacuum pressures.

According to an embodiment of any paragraph(s) of the present disclosure, the molded pulp material has optimized fiber length, fiber orientation, binder, and wall thickness such that the funnel surfaces are both air permeable and water resistant.

According to an embodiment of any paragraph(s) of the present disclosure, the molded pulp material is biodegradable.

According to an embodiment of any paragraph(s) of the present disclosure, the molded pulp material is compostable.

According to an embodiment of any paragraph(s) of the present disclosure, an inside surface of the urination funnel is smoother than an outside surface of the urination funnel.

According to an embodiment of any paragraph(s) of the present disclosure, the urination funnel further includes an air vent hole extending through the contoured body on a top side of the contoured body.

According to an embodiment of any paragraph(s) of the present disclosure, the urination funnel is configured to operate with a regulated value of suction within the range of 10-20 cmHg.

According to an embodiment of any paragraph(s) of the present disclosure, the urination funnel further includes at least one surface feature on a surface of the contoured body of the urination funnel.

According to an embodiment of any paragraph(s) of the present disclosure, the outlet is configured to directly engage with the suction hose of the suction canister system.

According to an embodiment of any paragraph(s) of the present disclosure, the outlet is configured to engage with the suction hose of the suction canister system via a connector.

According to another aspect of the present disclosure, a method of using a urination funnel for receiving and transporting urine voided by a user into a suction canister system is provided. The method includes the steps of connecting a suction hose of the suction canister system to an outlet of the urination funnel to form a hose seal between the urination funnel and the suction hose, positioning the urination funnel against an anatomy of the user such that an inlet of the urination funnel covers at least a urethra of the user, and activating the suction canister system when a patient is ready to urinate, such that urine is pulled into the urination funnel, then into the suction hose, and finally into a suction chamber of the suction canister system.

### BRIEF DESCRIPTION OF THE DRAWINGS

FIG. 1 is a perspective view of a urination funnel, showing its inlet, outlet opening and general shape.

FIG. 2 is another perspective view of the urination funnel, showing the major and minor axes and related planes of the inlet.

FIG. 3 is a side view of the urination funnel, showing the rolled lip and general shape of the contours.

FIG. 4 is another side view of the urination funnel, with a cross-sectional view of a suction hose connection to the urination funnel.

FIG. 5 is another perspective view of the urination funnel, showing a vent hole of the urination funnel.

FIG. 6A is a cross-sectional view of the urination funnel positioned over an individual in a supine position.

FIG. 6B is a cross-sectional view of the urination funnel positioned over an individual in a squatting or standing position.

FIG. 6C is a cross-sectional view of the urination funnel positioned over an individual in a seated position.

FIG. 7 is a schematic diagram showing the urination funnel connected to a suction canister system with a suction canister which is then connected to a suction source, with a suction hose.

FIG. 8 is a side view of the urination funnel having controlled surface features.

### DETAILED DESCRIPTION

Embodiments of the present application will now be described with reference to the drawings, wherein like reference numerals are used to refer to like elements throughout. It will be understood that the figures are not necessarily to scale. In the following description, numerous details and alternatives are set forth for purpose of explanation. However, one of ordinary skill in the art will realize that the invention can be practiced without the use of these specific details. For instance, the figures and description below often refer to the vulvar area of female anatomy and compostable materials. In other instances, however, well known structures and devices are shown in block diagram form in order not to obscure the description of the invention with unnecessary detail. In addition, one of ordinary skill in the art will recognize that some embodiments of the invention are practiced for urination by larger, or smaller, patients, and may include modifications for the male anatomy.

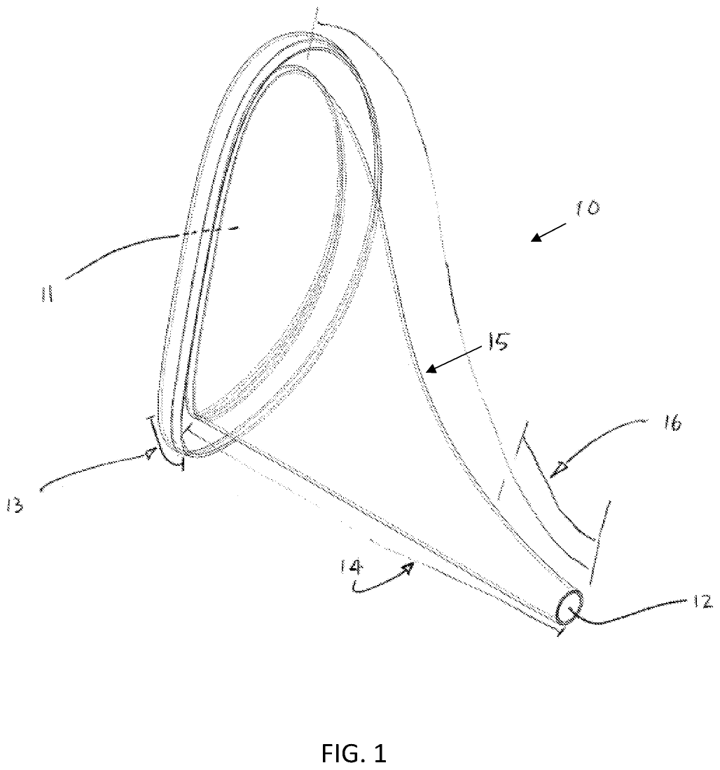

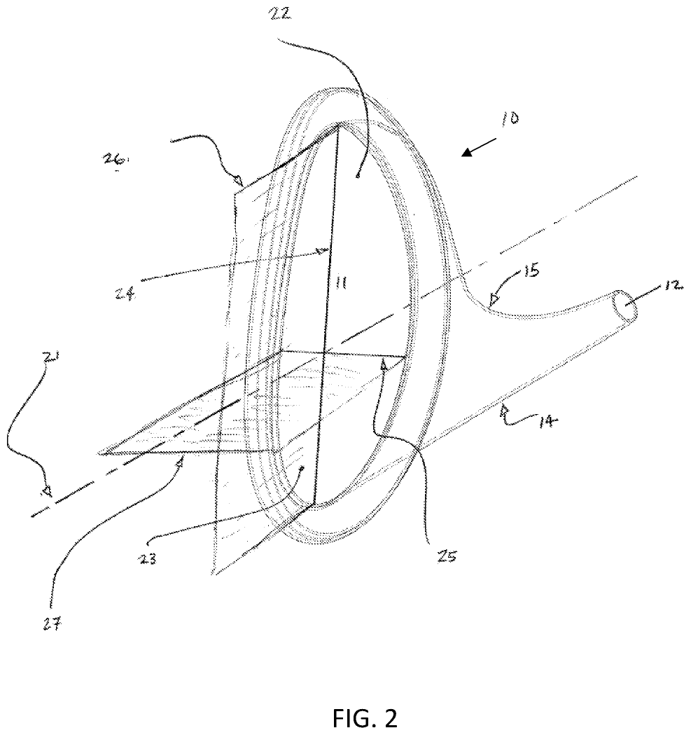

FIGS. 1-5 depict a urination funnel 10 having an elongated tube shape, which necks down from a larger end with an inlet (entrance) 11 to a smaller end with an outlet (exit) 12. As best depicted in FIG. 2, the inlet 11 is shaped like an ellipse with a major axis plane 26, defined by a major axis 24 and a top-bottom centerline 21, and a minor axis plane 27, defined by a minor axis 25 and the top-bottom centerline 21. The top-bottom centerline 21 extends through the center of the inlet 11 and is parallel to a straight portion 14 along a bottom of the urination funnel 10. Inside of the urination funnel 10, an inside top portion 22 is above the top-bottom centerline 21 and the minor axis plane 27, while an inside bottom portion 23 is below the top-bottom centerline 21 and the minor axis plan 27. The minor axis plane 27 is perpendicular to the major axis plane 26. The outlet 12 is cylindrical with a slight taper (circular in cross-section). The inlet 11 may have a dimension along the major axis 24 in the range of 75.0 mm to 125.0 mm, for example 90.0 mm, and may have a dimension along the minor axis 25 in the range of 25.0 mm to 75.0 mm, for example 38.5 mm.

Between the inlet 11 and the outlet 12, the body of the urination funnel 10 is lofted (contoured). The lofting/contour is not symmetric to the minor axis plane 27 through the minor axis 25 of the ellipse of the inlet 11 and along the length of the urination funnel 10. That is, as best shown in FIG. 1, the lofting asymmetry includes a top side that has a curved portion 15 and ends in a tapered section 16 starting from the inlet 11 and traveling along the length of the funnel to the outlet 12. A bottom of the urination funnel 10 includes the straight portion 14 which forms the bottom of the tapered section 16. The urination funnel 10 is mirror-image symmetric about the major axis plane 26.

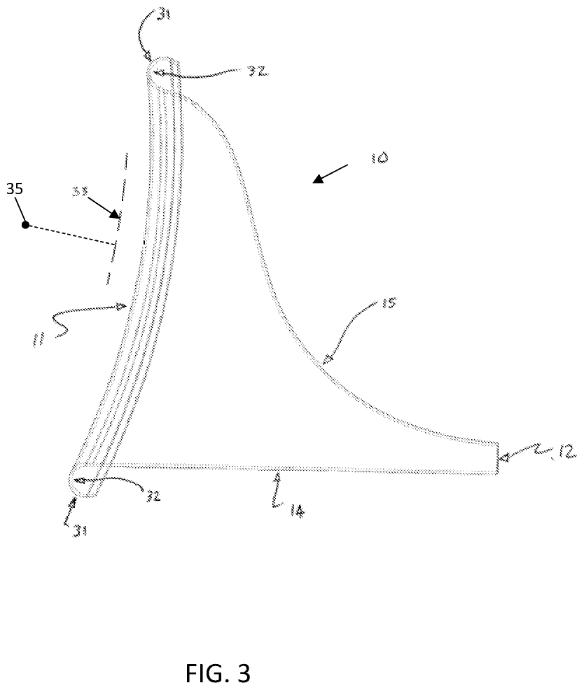

A side view of the urination funnel 10 is presented in FIG. 3. As depicted, the inlet 11 includes a rolled lip 13. The rolled lip 13 may be made of the same or different material as the body of the urination funnel 10 and extends from the curved portion 15 radially outwardly from the inlet 11, so as to extend radially outward and wider than the inlet 11. The rolled lip 13 may then curve and change direction so that the rolled lip 13 finishes generally 180 degrees from where it started from the curved portion 15. An inside of the rolled lip 32 comes from the curved portion 15 and curves around and ends in an outside of the rolled lip 31 which nearly reverses direction. The diameter of the rolled lip 31, in cross-section as depicted in FIG. 3, may be approximately 10.0 mm and may range from anywhere between 3.0 mm to 15.0 mm. It is understood that this diameter may be larger or smaller, depending on preference and patient sizing. The rolled lip 13 includes a curve 33, schematically depicted with a dashed line in FIG. 3. The curve 33 is shaped so as to fit comfortably on the anatomy of the user. For example, the curvature of the curve 33 may define a circle having a radius, relative to a center point 35, of 165.0 mm, or in a range between 135.0 mm to 195.0 mm. It is understood, however, that those with knowledge in fitting more specific anatomy can modify the shape or curvature of the curve 33, or the location of the rolled lip 13, for a better fit. That is, the curvature and/or location of the curve 33 (the vertex) can be changed, and can include features that allow the tapered section 16 of the urination funnel 10 to move up, down, in, and out with respect to the anatomy.

The shape of the rolled lip 13 may be different in various embodiments. For example, the shape of the rolled lip 13 may include a bead along the edge of the urination funnel 10 at the inlet 11. Additionally, the rolled lip 13 may be made of another material such as a closed or open foam, a softer material than the body of the urination funnel 10, or any combination thereof, for providing more comfort, better frictional characteristics, and usability.

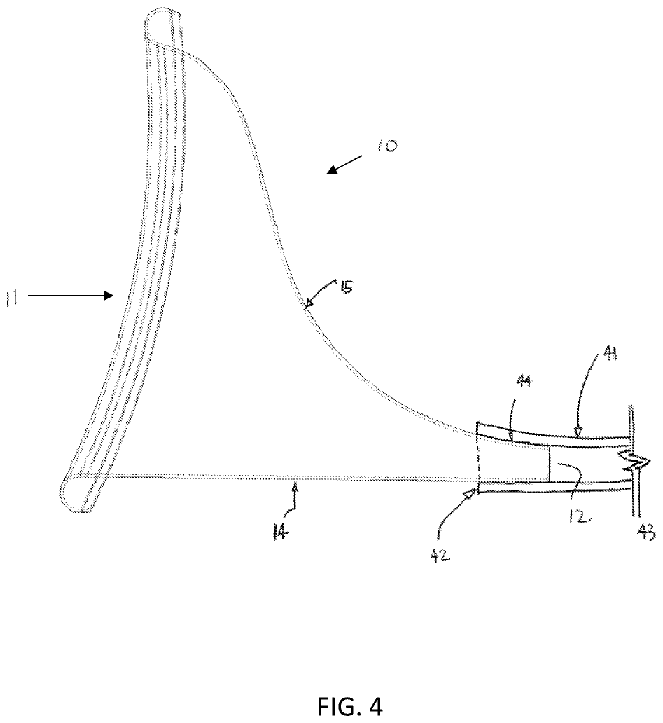

FIG. 4 depicts a suction hose 41 attached to the tapered section 16. In more detail, the suction hose 41 includes a suction hose attachment end 42 that is pushed to the left from the right over the outlet 12 and up onto the tapered section 16. A hose seal 44 is therefore formed between the tapered section 16 and the suction hose 41. As will be understood, the other end of the hose goes to either a drain for the urine, a canister for containing the urine, or some other receptacle to hold the urine until disposal, and then to the vacuum source which creates the suction. The length of the suction hose 41 is long enough for the system to work for a particular patient application. The urine receptacle and vacuum source may be standard items in a hospital, clinic, healthcare facility, or other location, such as a patient's home, and are therefore not shown. The suction hose break 43 indicates that the hose continues onto the other parts.

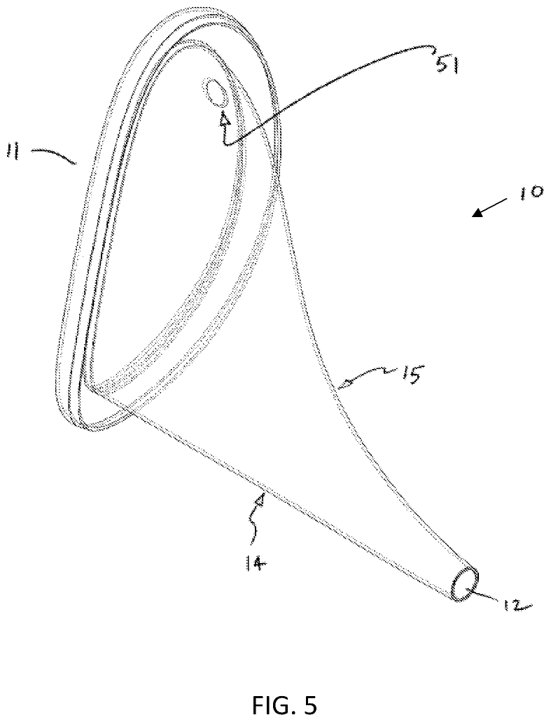

As depicted in FIG. 5, the urination funnel 10 may include a vent hole 51, shown near the inlet 11 and on the curved portion 15. The vent hole 51 may be used if the material of the urination funnel 10 does not afford enough air flow through it, or if additional vacuum control is desired by the patient or caregiver via adjusting partial coverage of the vent hole 51 during operation.

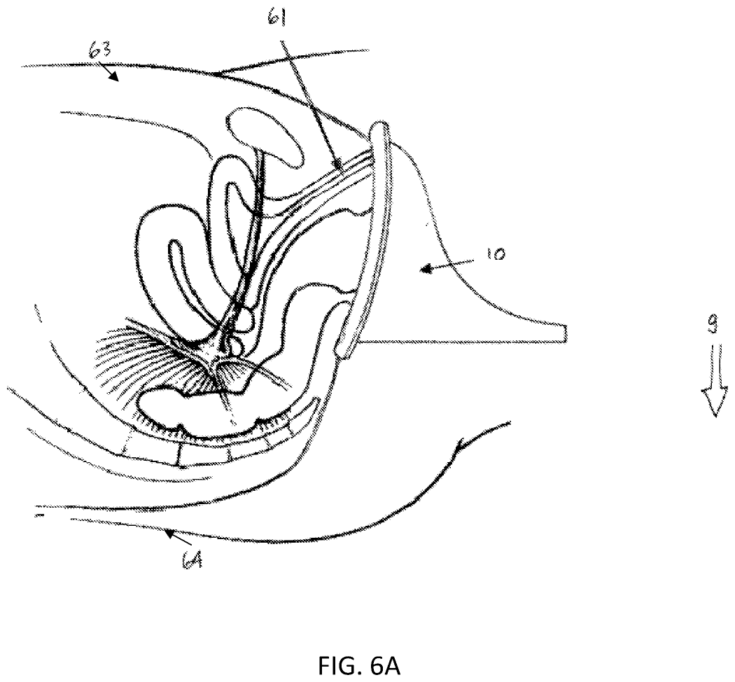

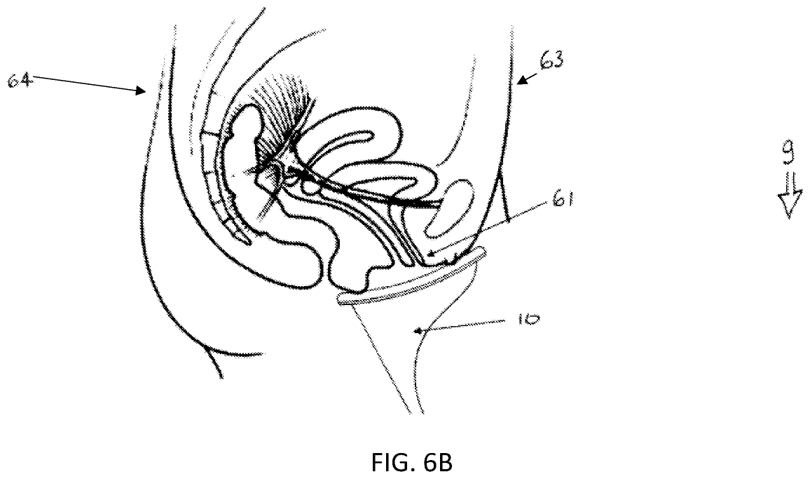

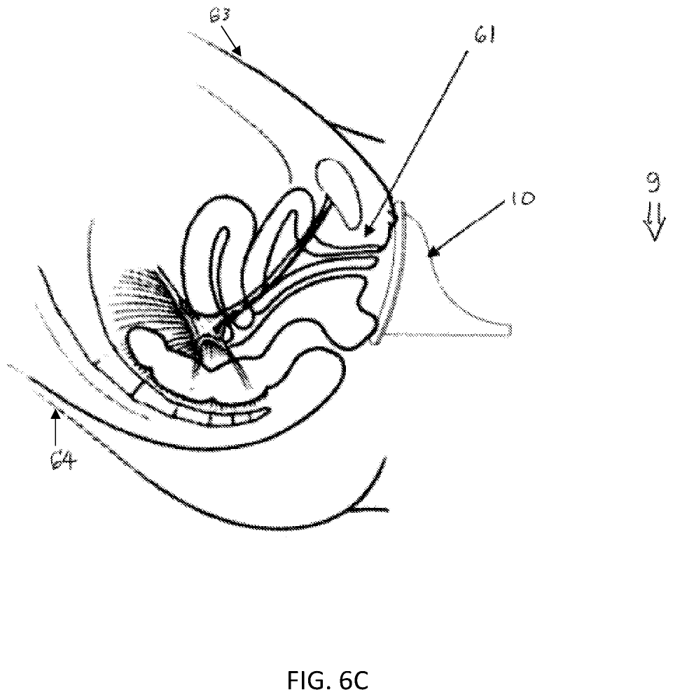

FIGS. 6A-6C depict views of the urination funnel 10 being used on a patient in various positions. Specifically, FIG. 6A depicts the urination funnel 10 used on a patient in a supine position (anterior side 63 up and posterior side 64 supported from below), FIG. 6B depicts the urination funnel 10 used on a patient in a standing or squatting position, and FIG. 6C depicts the urination funnel 10 used on a patient in a seated position. In the supine position of FIG. 6A, the position of the upper legs of the patient is suggested to be flexed at the hips, but this is not necessary. The arrow g represents the direction of gravity and provides an orientation reference for the three views. Suction through the system and a good fit to the patient affords a wide range of patient use positions. In each position depicted in FIGS. 6A-6C, the urination funnel 10 is oriented such that a urethra 61 of the patient is covered completely and such that the urination funnel 10 points generally towards the anterior side 63 of the patient.

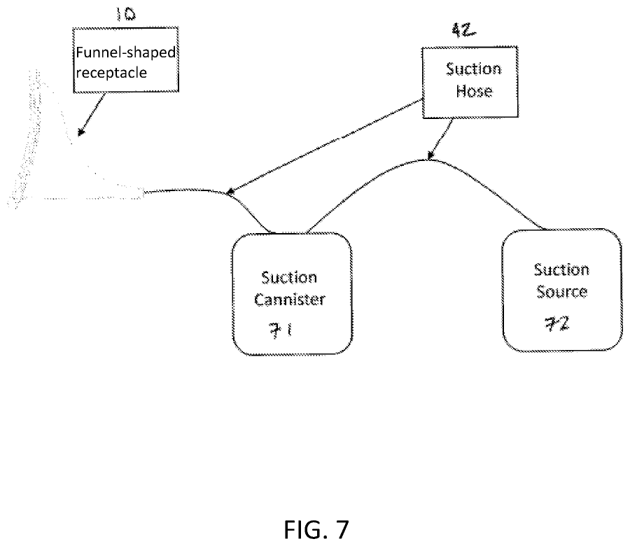

FIG. 7 is a schematic diagram representing the urination funnel 10 connected to a preferred removal system composed of a suction canister 71 and suction source 72, all connected with suction hoses 42. The suction canister 71 could be a bag, or other containment device, instead of a canister while still allowing for connection to the suction source 72. The size of the urination funnel 10 is designed to fit medical device standard tubing as defined in ISO 10079-4:2021(en) Medical suction equipment—Part 4: General requirements.

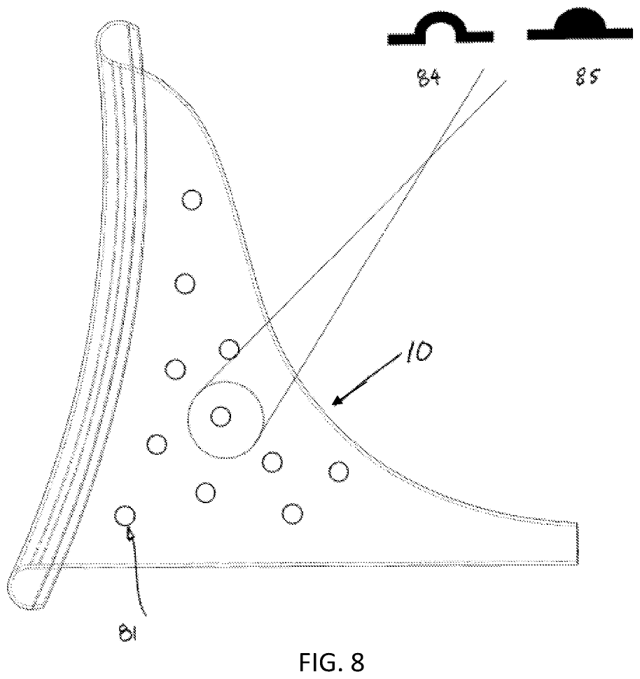

FIG. 8 depicts how the material surface of the urination funnel 10 can be modified to provide various patterns on the surface thereof. For example, these patterns may be formed as bumps 81 on the surface. The bumps 81 can be pushed out from, or added to, the material surface of the urination funnel 10 during the manufacture of the urination funnel 10. Alternatively, the bumps 81 may be added to the material surface of the urination funnel 10 after manufacture thereof using many different methods and materials. The bumps 81 are depicted in the exploded cross-sectional view of FIG. 8 (a view of the bump 81 if it were “cut-through” and viewed from the edge of the cut) in two types. For example, the bumps 81 may be an embossed bump 84 or a raised bump 85. The embossed bump 84 is pushed out by a proud feature on the inside portion of a mold and an indented feature on the outside portion of the mold. The raised bump 85 only has the outside indented feature of the mold. In other embodiments, other surface features and patterns may be provided which can customize the appearance of the urination funnel 10 and can provide three-dimensional shapes for different grips. These surface features may be accomplished by providing embossing in the mold.

In any embodiment having such bumps 81 or surface features, the bumps 81 or other surface features allow easy removal from stacking multiple urination funnels 10 together, and reduces the likelihood of two urination funnels 10 sticking to each other during transport and storage. The bumps 81 or other surface features do not have to be circular or located at the positions depicted in FIG. 8, nor do they need to keep multiple urination funnels 10 from sticking together. Many designs can be envisioned which would allow potentially better gripping by the patient, branding by the company, instructive 3D graphics all to be added to the surface.

The urination funnel 10 may be made from a molded pulp material. The fiber orientation, length, thickness of the pulp material (ranging from 0.25 mm to 5.0 mm, for example from 0.8 mm to 1.2 mm), the processing, the water-resistant waxes and binders all combine such that the stiffness of the urination funnel 10 does not collapse under normal handling and vacuum pressure while still maintaining control of the urination funnel 10. Also, the stiffness (rigidity) of the urination funnel 10 is high enough that the tapered section 16 does not collapse the outlet 12, nor collapse, or fold the tapered section 16 back on the middle of the urination funnel 10. In other embodiments, various configurations of material which combine to make various types of moldable pulp may be used. Some of these enable the ability to adjust the properties of water absorption, change the stiffness of the pulp, and other processing factors known in the industry.

The process of making pulp is essentially putting a pulp slurry (pulp, water, waxes, binders, and other process materials) around one side of a mold and then pressing a mesh, shaped like the other mold side, against the first side, heating the mold and drying out the water. This leaves one side (e.g., an inner side) smoother than the other side (e.g., an outer side), as the other side has an imprint of the mesh with a consistent roughness. This affords using the smooth side for the inside (from FIG. 2, the combination of inside top portion 21 and inside bottom portion 23) as well as the continuation of the rolled lip 13 which contacts the patient. The outer surface of the funnel is where the patient, or caregiver, grips the urination funnel 10 and this is the mesh surface. The rough surface acts to make the gripping of the device easier and requires less grip force to hold and control the device. As mentioned earlier bumps 81 can be placed on the outer surface to enhance the gripping as well as other shapes and designs.

In another embodiment the material for the urination funnel could be a mycelium-based fungus. Alternate embodiments include other polymeric materials which can be broken down under special conditions and some which are recyclable. Other plastics, which are not compostable, biodegradable, or recyclable, could be used, as could as could various metals.

The urination funnel 10 may be water-resistant for a period of time longer than a typical urination so that if it is not disposed of immediately, it will not breakdown in the interim. The molded pulp formulation is such that the funnel will breakdown under standard biodegradable and compostable conditions, for example it meets the BPI standards set for commercial compostability certification (https://bpiworld.org/compostability-certification).

A method of using the urination funnel 10 disclosed herein will now be described. The method includes removing the urination funnel 10 from a storage location and gripping, in one hand, the body of the urination funnel 10 around the curved portion 15. The method includes gripping, with another hand, the suction hose 41 and connecting the suction hose 41 to the outlet 12 of the urination funnel 10 by pushing the suction hose attachment end 42 of the suction hose 41 over the tapered section 16 of the urination funnel 10 until the suction hose 41 has covered a sufficient length (approx. 1 cm.) of the tapered section 16 to form the hose seal 44 and create a good seal in the hose seal 44 area.

The other end of the suction hose 41 is assumed to be already attached to an inlet port on a standard suction canister 71 which is part of a standard suction system, suction source 72 (either a facility-wide pressure regulated vacuum system or portable vacuum pump commonly used with suction canister systems). The sizes and configurations of these parts is specified by an international medical device standard, ISO 10079-4:2021(en) Medical suction equipment—Part 4: General requirements, ensuring the connections work in all situations, facilities, and equipment available.

Before activating the suction system, the patient assumes a comfortable position either laying down (supine), sitting up, or at any position in between. The method includes positioning the urination funnel 10 against an anatomy of the patient such that the inlet 11 of the urination funnel 10 covers at least a urethra of the user. As depicted in FIG. 6A-6C, when appropriately positioned, the curved section 15 of the urination funnel 10 is oriented towards the anterior 63 side of the patient's body and the rolled lip 13 is lightly pressed against their vulva. The urethra 61 is covered and the labia minor will tuck into the urination funnel 10, but the entire vulva does not need to be covered for the urination funnel 10 to operate. The best results are when the urethra is positioned within the area defined as the inside top portion 22 of the urination funnel 10.

The method then includes activating the suction system when a patient is ready to urinate. The suction, or vacuum, system is set to a low setting, typically 10-15 cm Hg. The present invention has been operationally tested at vacuum pressures between 7.5-cmHg with good outcomes. The vacuum level adjustment allows for patient preference under some circumstances and material considerations of pulp (air can flow through the pulp and the rate depends on many of the pulp's material characteristics).

As the patient is ready to urinate and the suction system is activated, the patient will feel a gentle suction and the system is ready to use. The patient begins to urinate. The urine is pulled into the urination funnel 10, then into the suction hose 41, and finally into the suction canister. Once the patient finishes urinating, the urination funnel is left in place for 5-10 seconds ensuring all the urine is removed and then is slowly removed from the vulva and this portion of the process is complete.

The method then includes removing the urination funnel 10 from the patient. The best method for removal is to slowly tip the curved portion 15 of the urination funnel 10 away from the body of the patient first and then pull the rest of the urination funnel 10 away from the body of the patient. At this time the urination funnel 10 is grasped by one hand in the same manner as before, with the suction hose 41 in the other hand, and with a slight twist and pull apart the two are separated. The suction hose 41 is cleaned and stored for the next use while the urination funnel is placed into a composting system for disposal and composting.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A urination funnel for receiving and transporting urine voided by a user into a suction canister system, the urination funnel comprising:
an inlet for receiving the urine voided by the user, wherein the inlet is configured to conform in shape to an anatomy of the user around a urethra of the user;
an outlet for transporting the urine voided by the user to the suction canister system, wherein the outlet is configured to engage with a suction hose of the suction canister system; and
a contoured funnel body extending between the inlet and the outlet of the contoured funnel body.

2. The urination funnel according to claim 1, wherein the inlet includes a rolled lip that is configured to conform in shape to the anatomy of the user around the urethra of the user.

3. The urination funnel according to claim 1, wherein a material of the urination funnel is a molded pulp material from plant-based fiber material.

4. The urination funnel according to claim 3, wherein the plant-based fiber material is recycled.

5. The urination funnel according to claim 3, wherein the molded pulp material has optimized fiber length, fiber orientation, binder, and wall thickness to provide sufficient structural rigidity to support low vacuum pressures.

6. The urination funnel according to claim 3, wherein the molded pulp material has optimized fiber length, fiber orientation, binder, and wall thickness such that the funnel surfaces are both air permeable and water resistant.

7. The urination funnel according to claim 3, wherein the molded pulp material is biodegradable.

8. The urination funnel according to claim 3, wherein the molded pulp material is compostable.

9. The urination funnel according to claim 3, wherein an inside surface of the urination funnel is smoother than an outside surface of the urination funnel.

10. The urination funnel according to claim 1, further comprising an air vent hole extending through the contoured body on a top side of the contoured body.

11. The urination funnel according to claim 1, wherein the urination funnel is configured to operate with a regulated value of suction within the range of 10-20 cmHg.

12. The urination funnel according to claim 1, further comprising at least one surface feature on a surface of the contoured body of the urination funnel.

13. The urination funnel according to claim 1, wherein the outlet is configured to directly engage with the suction hose of the suction canister system.

14. The urination funnel according to claim 1, wherein the outlet is configured to engage with the suction hose of the suction canister system via a connector.

15. A method of using a urination funnel for receiving and transporting urine voided by a user into a suction canister system, the method comprising the steps of:
connecting a suction hose of the suction canister system to an outlet of the urination funnel to form a hose seal between the urination funnel and the suction hose;
positioning the urination funnel against an anatomy of the user such that an inlet of the urination funnel covers at least a urethra of the user; and
activating the suction canister system when a patient is ready to urinate, such that urine is pulled into the urination funnel, then into the suction hose, and finally into a suction chamber of the suction canister system.
